(19) United States
(12) Patent Application Publication
(10) Pub. No.: US 2008/0058640 A1
(43) Pub. Date: Mar. 6, 2008

(54) Marker formed of starch or other suitable polysaccharide

(75) Inventors: Michael L. Jones, San Clemente, CA (US); Paul Lubock, Laguna Niguel, CA (US)

Correspondence Address: EDWARD J. LYNCH;Patent Attorney, Suite 562, One Embarcadero Center, San Francisco, CA, 94111, US

(73) Assignee: SenoxRx, Inc.

(21) Appl. No.: 11/980,291

(22) Filed: Oct. 30, 2007

Related U.S. Application Data
(62) Division of application No. 11/881,264, filed on Jul. 26, 2007.
(60) Provisional application No. 60/835,740, filed on Aug. 4, 2006.

(51) Int. Cl. A61B 6/00

(52) U.S. Cl. 600/432; 600/431; 600/435

(57) ABSTRACT

The marker member delivery system described has at least one marker member disposed within a delivery tube or cannula is formed at least in part of starch or a suitable polysaccharide. The marker member is preferably in the form of fibrous member.

### RELATED APPLICATIONS

This application is related to and claims priority from provisional application Ser. No. 60/835,740, filed on Aug. 4, 2006, which is incorporated herein by reference in its entirety.

### FIELD OF THE INVENTION

The invention is generally directed to remotely detectable, intracorporeal markers and devices and methods for the delivery of such markers to a desired location within a patient's body.

### BACKGROUND OF THE INVENTION

In diagnosing and treating certain medical conditions, it is often desirable to mark a suspicious body site for the subsequent taking of a biopsy specimen, for delivery of medicine, radiation, or other treatment, for the relocation of a site from which a biopsy specimen was taken, or at which some other procedure was performed. As is known, obtaining a tissue sample by biopsy and the subsequent examination are typically employed in the diagnosis of cancers and other malignant tumors, or to confirm that a suspected lesion or tumor is not malignant. The information obtained from these diagnostic tests and/or examinations is frequently used to devise a therapeutic plan for the appropriate surgical procedure or other course of treatment.

In many instances, the suspicious tissue to be sampled is located in a subcutaneous site, such as inside a human breast. To minimize surgical intrusion into a patient's body, it is often desirable to insert a small instrument, such as a biopsy needle, into the body for extracting the biopsy specimen while imaging the procedure using fluoroscopy, ultrasonic imaging, x-rays, magnetic resonance imaging (MRI) or any other suitable form of imaging technique or palpation. Examination of tissue samples taken by biopsy is of particular significance in the diagnosis and treatment of breast cancer. In the ensuing discussion, the biopsy and treatment site described will generally be the human breast, although the invention is suitable for marking biopsy sites in other parts of the human and other mammalian body as well.

Periodic physical examination of the breasts and mammography are important for early detection of potentially cancerous lesions. In mammography, the breast is compressed between two plates while specialized x-ray images are taken. If an abnormal mass in the breast is found by physical examination or mammography, ultrasound may be used to determine whether the mass is a solid tumor or a fluid-filled cyst. Solid masses are usually subjected to some type of tissue biopsy to determine if the mass is cancerous.

If a solid mass or lesion is large enough to be palpable, a tissue specimen can be removed from the mass by a variety of techniques, including but not limited to open surgical biopsy, a technique known as Fine Needle Aspiration Biopsy (FNAB) and instruments characterized as “vacuum assisted large core biopsy devices”.

If a solid mass of the breast is small and non-palpable (e.g., the type typically discovered through mammography), a vacuum assisted large core biopsy procedure is usually used. In performing a stereotactic biopsy of a breast, the patient lies on a special biopsy table with her breast compressed between the plates of a mammography apparatus and two separate x-rays or digital video views are taken from two different points of view. A computer calculates the exact position of the lesion as well as depth of the lesion within the breast. Thereafter, a mechanical stereotactic apparatus is programmed with the coordinates and depth information calculated by the computer, and such apparatus is used to precisely advance the biopsy needle into the small lesion. The stereotactic technique may be used to obtain histologic specimens. Usually at least five separate biopsy specimens are obtained from locations around the small lesion as well as one from the center of the lesion.

The available treatment options for cancerous lesions of the breast include various degrees of mastectomy or lumpectomy, radiation therapy, chemotherapy and combinations of these treatments. However, radiographically visible tissue features, originally observed in a mammogram, may be removed, altered or obscured by the biopsy procedure, and may heal or otherwise become altered following the biopsy. In order for the surgeon or radiation oncologist to direct surgical or radiation treatment to the precise location of the breast lesion several days or weeks after the biopsy procedure was performed, it is desirable that a biopsy site marker be placed in the patient's body to serve as a landmark for subsequent location of the lesion site. A biopsy site marker may be a permanent marker (e.g., a metal marker visible under x-ray examination), or a temporary marker (e.g., a bioresorbable marker detectable with ultrasound). While current radiographic type markers may persist at the biopsy site, an additional mammography generally must be performed at the time of follow up treatment or surgery in order to locate the site of the previous surgery or biopsy. In addition, once the site of the previous procedure is located using mammography, the site must usually be marked with a location wire which has a hook on the end which is advanced into site of the previous procedure. The hook is meant to fix the tip of the location wire with respect to the site of the previous procedure so that the patient can then be removed from the confinement of the mammography apparatus and the follow-up procedure performed. However, as the patient is removed from the mammography apparatus, or otherwise transported the position of the location wire can change or shift in relation to the site of the previous procedure. This, in turn, can result in follow-up treatments being misdirected to an undesired portion of the patient's tissue.

As an alternative or adjunct to radiographic imaging, ultrasonic imaging (herein abbreviated as “USI”) or visualization techniques can be used to image the tissue of interest at the site of interest during a surgical or biopsy procedure or follow-up procedure. USI is capable of providing precise location and imaging of suspicious tissue, surrounding tissue and biopsy instruments within the patient's body during a procedure. Such imaging facilitates accurate and controllable removal or sampling of the suspicious tissue so as to minimize trauma to surrounding healthy tissue.

For example, during a breast biopsy procedure, the biopsy device is often imaged with USI while the device is being inserted into the patient's breast and activated to remove a sample of suspicious breast tissue. As USI is often used to image tissue during follow-up treatment, it may be desirable to have a marker, similar to the radiographic markers discussed above, which can be placed in a patient's body at the site of a surgical procedure and which are visible using USI. Such a marker enables a follow-up procedure to be performed without the need for traditional radiographic mammography imaging which, as discussed above, can be subject to inaccuracies as a result of shifting of the location wire as well as being tedious and uncomfortable for the patient.

Placement of a marker or multiple markers at a location within a patient's body requires delivery devices capable of holding markers within the device until the device is properly situated within a breast or other body location. Accordingly, devices and methods for retaining markers within a marker delivery device while allowing their expulsion from the devices at desired intracorporeal locations are desired.

In addition to marking functions, frequently it is desirable to provide treatments with the marker members such as hemostatic treatment and the like.

### SUMMARY OF THE INVENTION

The invention is generally directed to a remotely imageable marker system suitable for deployment at a site within a patient's body, particularly a biopsy site such as in a patient's breast. The imageable marker system is formed of a plurality of marker members with at least one of the marker members containing starch or other polysaccharide sufficient to accelerate thrombus formation at the site where tissue has been removed. The starch or other suitable polysaccharide has a molecular weight of about 3500 to about 200,000 Daltons and is preferably formed from a dry powder thereof having a particle size of about 20-100 micrometers. The marker member with starch or other suitable polysaccharide may be a pellet formed of compressed starch powder which may be included in a gelatin capsule that controls release timing. Alternatively, the marker member with starch or other suitable polysaccharide may be in the form of a fibrous pad or mat, e.g. felt, formed of strands of synthetic polymeric material with a powder of starch or other suitable polysaccharide incorporated into the pad or mat or rolled up or folded up with the pad or mat. These small particles of starch or other polysaccharide rapidly absorb fluid and hydrate and in the process dehydrate blood at the site of deployment to rapidly initiate clotting.

The marker body may also include a radiopaque element connected thereto or incorporated therein. The radiopaque element provides long term identification of the intracorporeal site. Preferably, the radiopaque element is formed of non-magnetic material to avoid interference with magnetic resonance imaging (MRI). Suitable non-magnetic materials include titanium, platinum, gold, iridium, tantalum, tungsten, silver, rhodium, non-magnetic stainless steel (316) and the like. The radiopaque element should have a non-natural shape so that it is readily recognized when remotely imaged. The radiopaque element should have a maximum dimension of about 0.5 to about 5 mm, preferably about 1 to about 3 mm to ensure remote identification, particularly with MRI.

The pellets will generally be about 0.2 to about 3 mm, preferably about 1 to about 2 mm, in diameter and about 3 to about 7 mm, preferably about 4 to about 6 mm in length. The fibrous body of the marker member is formed into a an elongated member suitable for delivery by rolling or folding a fibrous mat or pad, and binding the rolled or folded mat or pad in a compressed condition to provide sufficient column strength to facilitate introduction into and discharge of the compressed and rolled body from a tubular delivery device. Suitable binding agents for holding the fibrous marker in a compressed condition are water soluble polymers such as polyvinyl alcohol, polyethylene glycol and polyvinyl pyrollidone. The fibrous pad or mat preferably has a bulk density greater than 10 mg/cc, preferably about 30 to about 100 mg/cc. Preferably, the fibrous pad or mat is formed of oxidized cellulose the synthetic polymer strands are hydrophobic. Suitable commercially available felt matting has a bulk density of about 40 mg/cc. Synthetic polymeric materials which are predominantly polyglycolic acid (PGA), i.e. at least 50% (by weight) or other synthetic polymeric materials such as polylactic acid, polycaprolactone and copolymers thereof and therewith.

The marker member embodying features of the invention can be readily delivered to the desired location by suitable delivery systems such as disclosed in co-pending applications Ser. No. 10/444,770, filed on May 23, 2003 and Ser. No. 10/753,277, filed on Dec. 23, 2003. The marker delivery system generally has an elongated cannula or syringe-like body with proximal and distal ports and an inner lumen extending between the ports. The marker member is slidably disposed within the inner lumen of the delivery cannula and a plunger slidably disposed within the inner lumen of the delivery cannula proximal to the markers. The plunger is movable from an initial position proximal to the markers within the tube, to a delivery position close to the discharge opening in the distal end of the cannula to push the marker members out of the discharge opening into the target tissue site.

Upon being discharged into the intracorporeal target site, the starch or other suitable polysaccharide quickly takes up body fluid at the site initiating the clotting process. The marker member at least partially fills the site to enable short term detection (at least three weeks, preferably at least four weeks but less than a year) by remote USI and preferably long term detection by remote mammographic imaging or MRI identification. The binding agent in the marker body is dissolved so the fibrous body can expand within the intracorporeal site. While the marker body takes up water, the individual strands which form the marker preferably do not swell significantly (less than 5%) on contact with body fluid. The expanded fibrous marker positions the radiopaque marker element within the interior of the target cavity.

The cannula of the marker delivery device may be configured to fit within the guide cannula of a biopsy device, such as a Mammotomee (sold by Johnson & Johnson), the SenoCor 360™ biopsy device sold by SenoRx (the present assignee), the EnCor,™ biopsy device sold by SenoRx and or a coaxial needle guide. The delivery cannula can also be configured to fit into the proximal end of a tubular cutting element such as found in the EnCor™ biopsy system sold by SenoRx which is the subject of co-pending application Ser. No. 10/911,106, filed on Aug. 3, 2004.

One suitable delivery system suitable for delivery through a tubular cutter (e.g. as with the Oncore™ system) is a syringe-type delivery system described in co-pending application Ser. No. 10/911,106, filed on Aug. 3, 2004 having a tubular shaft with a flared guide on or integral with the distal tip to facilitate engagement with the proximal end of the tubular cutter. Another syringe-type delivery system has a plugged distal tip to prevent body fluids from engaging one or more markers which may be in the tubular shaft of the delivery system. Such fluid infusions can retard or restrict discharging the fibrous marker and other markers which may be within the inner lumen of the delivery cannula. Delivery systems with plugged tips are described in co-pending applications Ser. No. 10/444,770, filed on May 23, 2003 and Ser. No. 10/753,277, filed on Dec. 23, 2003, which are incorporated herein in their entireties. The plugged tip type delivery systems can have a side opening for marker deployment or a plugged needle-type distal tip both of which are disclosed in the above application Ser. No. 10/753,694.

A variety of therapeutic or diagnostic agents may be incorporated into the fibrous marker. Incorporated agents can include for example, hemostatic agents to form thrombus at the intracorporeal site, anesthetic agents to control pain, chemotherapeutic agents for treating residual neoplastic tissue or coloring agents to facilitate subsequent visual location of the site. Antibiotics, antifungal agents and antiviral agents may also be incorporated into the fibrous marker.

Upon delivery to the intracorporeal site, the fibrous marker unrolls and expands to facilitate identification and localization. The marker is easily identifiable from surrounding tissue at the site by ultrasonic imaging (USI).

The fibrous markers embodying features of the present invention provide several advantages. The synthetic polymeric strands are preferably hydrophobic which eases the difficulty in manufacturing the markers because they do not react with surrounding moisture. Moreover, the fibrous marker material is stabilized quickly in the intracavity clot which forms at the biopsy site and can be readily identified from surrounding tissue of the cavity by less skilled radiologists or surgeons.

These and other advantages of the invention will become more apparent from the following detailed description of embodiments when taken in conjunction with the accompanying exemplary drawings.

### BRIEF DESCRIPTION OF THE DRAWINGS

FIG. 1 is an elevational view of a fibrous marker member embodying features of the invention.

FIG. 2 is a perspective end view of the fibrous marker member shown in FIG. 1 illustrating the rolled structure of the marker member.

FIG. 3A is a partly cut-away perspective view of a marker delivery assembly for the fibrous marker member shown in FIG. 1.

FIG. 3B is a transverse cross-sectional view of the marker delivery assembly of FIG. 3A taken at line 3B-3B.

FIG. 3C is a transverse cross-sectional view of the marker delivery assembly of FIG. 3A taken at line 3C-3C.

FIG. 4 is a perspective view, partially in section, of a human breast from which a biopsy specimen has been removed, showing a fibrous marker being delivered to the biopsy site with the marker delivery assembly shown in FIG. 3A.

FIG. 5 is a partly cut-away perspective view of a marker delivery assembly similar to the delivery assembly shown in FIG. 3A for a plurality of marker members in pellet form.

FIG. 6 is a transverse cross-sectional view of a marker member having an outer surface or capsule.

### DETAILED DESCRIPTION OF EMBODIMENTS OF THE INVENTION

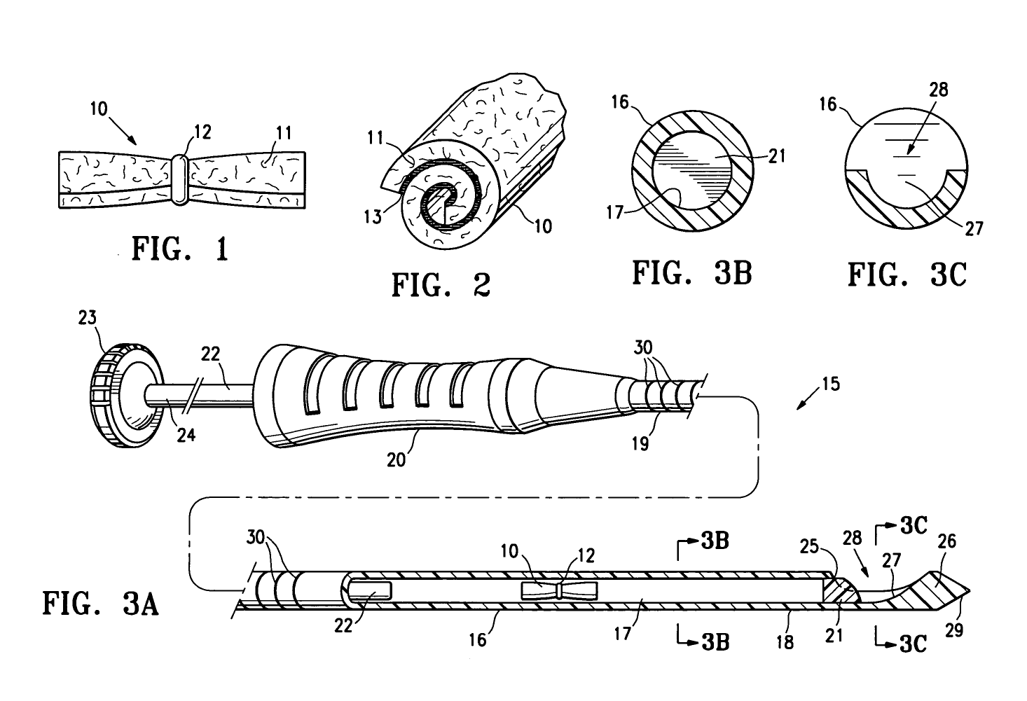

FIGS. 1 and 2 illustrate a fibrous marker member 10 embodying features of the invention. The fibrous marker member 10 is a rolled body formed of fibers or strands 11 with a radiopaque non-magnetic ring or wireform 12 encircling at least part of the central portion of the rolled body. The fiber or strands 11 are preferably formed of bioabsorbable synthetic polymeric material that is essentially hydrophobic and has an effective in-vivo life-span of at least three weeks, preferably at least four weeks. The fibrous marker member 10 is a rolled (or folded) and preferably compressed fibrous mat with binding material incorporated into the fibrous body to maintain the compressed condition. Starch powder 13 is incorporated into the upper surface of the fibrous mat prior to rolling so that the starch quickly forms thrombus when coming into contact with blood at the intracorporeal site. The rolled fibrous marker member 10 may be formed from a felt (as shown) or woven material. Preferably, the binding agent is a water soluble polymer such as polyethylene glycol which is incorporated into the fibrous marker member 11 and the body compressed to reduce the profile of the body and facilitate sliding the rolled body through a lumen of a marker delivery system to the desired site within the patient's body.

One suitable starch/polysaccharide material is Hemaderm™ which is available from Medafor, Inc. located in Minneapolis, Minn. This product is described at least in part in U.S. Pat. No. 6,060,461.

One suitable marker delivery system fibrous marker delivery system 15 is depicted in FIGS. 3A-3C which includes a delivery tube or cannula 16 with an inner lumen 17, a distal portion 18, and a proximal portion 19 with a handle 20. A releasable distal plug 21 and the fibrous marker 10 are shown disposed within the inner lumen 17. A plunger 22 is slidably disposed within the inner lumen 17 and is provided with a head 23 on the proximal end 24 configured to allow an operator to press the plunger further into the inner lumen and push both the releasable plug 21 and fibrous marker member 10 out of the discharge port or opening 25 in the distal end 26 of delivery cannula 16. Cannula handle 20 allows an operator to hold the cannula steady while pressing plunger 22 to discharge the releasable plug 21 and fibrous marker member 10.

Releasable plug 21 may substantially fill the discharge opening 25, as shown in FIG. 3A, or may occupy or block only a portion of the discharge opening. The exposed face of plug 21 is preferably provided with an inclined configuration. Releasable plug 21 is configured to be tight enough, e.g. press fit, in the inner lumen 17 to prevent its inadvertent release which might allow premature discharge of marker 10 from delivery cannula 16, but the plug must be easily released when the plunger 22 is pressed deeper into the inner lumen 17 of the delivery cannula 16. An adhesive or mechanical element(s) may be used to hold the releasable plug 21 in a position within the inner lumen 17 to occlude the discharge opening 25. Suitable adhesives include polyurethane or polyacrylic based adhesives, polyhydroxymethacrylate base adhesives, fibrin glue (e.g., Tisseal™), collagen adhesive, or mixtures thereof. Suitable mechanical means for securing the releasable plug 21 are described in co-pending application Ser. No. 10/174,401. The distal end 26 of the delivery cannula 16 is provided with a ramp 27 which guides the discharged plug 21 and marker member 10 out of the side port 28 into the target site. The distal tip 29 may be tapered for delivery through a guide tube (not shown).

The delivery cannula 16 may be provided with markings 30 which serve as visual landmarks to aid an operator in accurately placing the distal portion 18 of the cannula 16 in a desired location within a patient's body for discharging the marker 10.

The exterior of the delivery cannula 16 is preferably configured to fit within a guide cannula sized to accept a Mammotome®, Tru-Cut®, SenoCor® or EnCor™ biopsy device. Typically, plug 21 and marker member 10 will have diameters determined by the size of the inner lumen 17 and typically will be about 0.02 inch (0.5 mm) to about 0.5 inch (12 mm), preferably about 0.04 inch (1 mm) to about 0.3 inch (8 mm). Plug 21 may have slightly larger transverse dimensions to provide a tight fit.

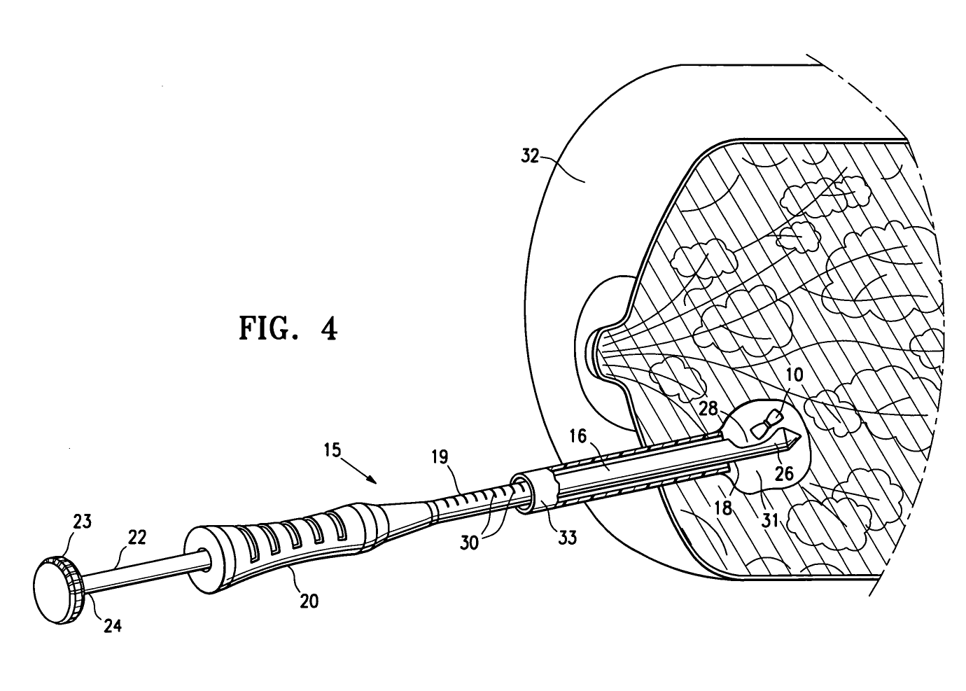

FIG. 4 schematically illustrates the delivery of fibrous marker member 10 to a cavity 31 such as a biopsy site in a patient's body. The distal portion of the cannula 16 marker delivery system 15 is shown inserted into a breast 32 through a guide cannula 33 until the distal end is disposed in the cavity 31 where a tissue specimen has been removed. While an operator holds the system 15 by the handle 20 of the delivery tube 16, the plunger 22 is pressed further into the bore 17 of delivery cannula 16 to discharge the releasable plug 21 and marker member 10 into the cavity 31. FIG. 4 schematically illustrates delivery of the marker member 10 within the cavity 31 after deployment. When the marker member 10 contacts body fluid within the cavity 31, the binding agent is dissolved and the fibrous body of the marker 10 expands to further fill the cavity 31. When blood at the intracorporeal site comes into contact with the starch or other polysaccharide incorporated into the fibrous body, moisture is drawn away from the blood and the clotting cascade begins to form thrombus at the site.

The fibrous marker member 10 is preferably a rolled or folded piece of fibrous mat formed of a bioresorbable synthetic polymeric material, preferably PGA which has been compressed and impregnated with a binding agent such as polyethylene glycol and freeze dried in the compressed condition. Alternatively, the fibrous mat forming the fibrous marker member 10 may be first compressed, rolled or otherwise shaped and then impregnated with binding agent and dried. The fibrous material may be rolled up by itself or wrapped about a core. The fibrous marker member 10 is generally about 0.5 mm to about 12 mm, preferably about 1 to about 8 mm in maximum transverse dimension and about 5 to about 30 mm, preferably about 10 to about 25 mm in length. Upon contact with a body fluid or other water based fluid, the length of the fibrous marker remains about the same but the wrapped structure unfolds due to the dissolution of the binding agent to a width of about 5 to about 25 mm, usually about 10 to about 20 mm. While the radiopaque marker ring 12 clamped about a center portion of the wrapped fibrous marker member 10, the fibrous marker unrolls or unfolds or otherwise expands when exposed to body fluids due to the dissolution of the binding agent which holds the marker in a compressed condition. However, even though secured to the fibrous marker member 10, the radiopaque marker ring 12 need not be surround the central portion of the marker as shown in the drawings, nor does it need to restrict the expansion of the fibrous marker as shown.

The manufacture of fibrous marker member 10 preferably starts with a fibrous mat of PGA with a bulk density of about 40 mg/mm and having a thickness of about 0.04 to about 0.375 inch (1-9.3 mm), preferably about 0.6 to about 0.8 inch (15.4-20.3 mm) thick. The mat is rolled, impregnated with a 30% (Wt.) polyethylene glycol in a 70% isopropyl alcohol solution and then compressed. The compressed and rolled mat is then freeze dried to a diameter of about 0.065 inch (1.65 mm). Elevated temperatures may be employed to dry the fibrous material. A radiographically detectable, non-magnetic marker ring 12 may be formed of wire about 0.005 to about 0.01 inch, (0.13-0.25 mm) may then be crimped about or embedded in a central portion (or other desired portion) of the rolled and compressed fibrous body to form the fibrous marker 10. The fibrous marker 10 is then ready for deployment. Suitable fibrous material is a felt mat sold as SCAFTEX by Biomedical Structures in Slatersville, R.I.

The size and composition of the strands of the fibrous marker member 10 and the bulk density are selected so that the fibrous marker is imageable in vivo by USI for at least 3 weeks, preferably about 4 to about 12 weeks for an effective lifespan. However, the fibrous marker member 10 should not be detectable by ultrasound after about one year, preferably not after about six months, so as to avoid interfering with subsequent site examination. The radiopaque and MRI detectable marker ring or element generally will have much longer lifespan, e.g. over a year.

The amount of starch or other suitable polysaccharide incorporated into the marker member 10 is sufficient to cause rapid clotting upon delivery to an intracorporeal site. Typically, the minimum amount of starch to water to form a suitable gel is at least 5%, preferably at least about 10% (wt %).

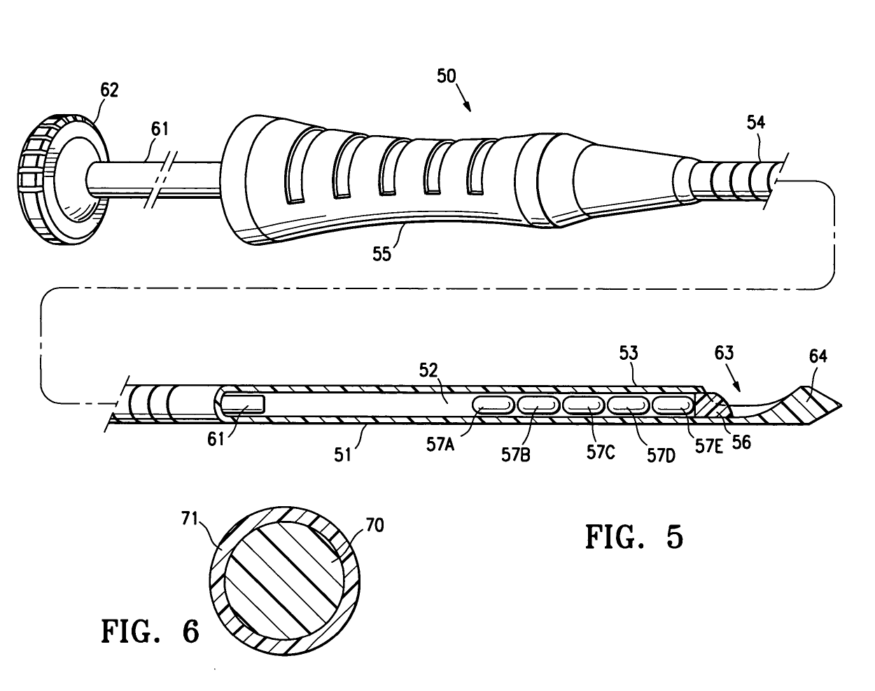

FIG. 5 illustrates a delivery system 50 embodying features of the invention is essentially the same system as shown in FIG. 3A. The system 50 includes a delivery tube or cannula 51 with a bore 52, a distal portion 53, and a proximal portion 54 with a handle 55. A releasable distal plug 56 and several (five) pellet style marker members 57A-E are shown disposed within the bore 52. A plunger 61 is slidably disposed within the tube bore 52, and is provided with a proximal end 62 configured to allow an operator to press the plunger further into the bore 52 and push the releasable plug 56 and one or more of the other marker members out of the discharge port or opening 63 in the distal end 64 of delivery tube 51. Cannula handle 55 allows an operator to hold the cannula steady while pressing plunger 61 to discharge the releasable plug 56 and marker members 57A-E. Marker members 57B and 57D are formed of starch powder which is pressed into the desired pellet shape. The other marker members 57A, C and E may be made of bioabsorbable material such as the bioabsorbable polymers described above. (See the description of marker pellets in co-pending applications Ser. No. 10/444,770, filed on May 23, 2003 and Ser. No. 10/753,277, filed on Dec. 23, 2003. Other marker members such as the fibrous marker member previously described may be disposed within the bore 52 along with the pellet shaped marker members. As shown in FIG. 6, the pellet shaped marker member 70 formed of starch or other suitable polysaccharide may have a protective coating 71 which retards taking up fluid until deployed well into the intracorporeal site. The coating may be a gelatin capsule.

Releasable plug 56 may substantially fill the discharge opening 63, as shown in FIG. 3A, or may occupy or block only a portion of the discharge opening 63. The exposed face of plug 56 is preferably provided with an inclined configuration to conform with the inclination of the discharge opening 63.

Marker members 57A-E are preferably configured to slide readily within tube bore 52. Releasable plug 56 is configured to be tight enough, e.g. press fit, in the bore 52 to prevent its inadvertent release which would allow premature discharge of marker members as previously described. An adhesive or mechanical element(s) may be used to hold the releasable plug 56 in a position within the bore 52 to occlude the discharge opening 63.

While one or more particular forms of the invention have been illustrated and described herein in the context of a breast biopsy site, it will be apparent that the device and methods having features of the invention may find use in a variety of locations and in a variety of applications, in addition to the human breast, where tissue has been removed. Moreover, various modifications can be made without departing from the spirit and scope of the invention. Accordingly, it is not intended that the invention be limited to the specific embodiments illustrated. It is therefore intended that this invention to be defined by the scope of the appended claims as broadly as the prior art will permit, and in view of the specification if need be. Moreover, those skilled in the art will recognize that features shown in one embodiment may be utilized in other embodiments. Additional details of pellet or fibrous marker members and delivery systems may be found in co-pending applications Ser. No. 10/753,694, filed on Jan. 7, 2004, and Ser. No. 10/976,138, filed on Oct. 27, 2004. Terms such as “element”, “member”, “device”, “section”, “portion”, “step”, “means” and words of similar import when used in the following claims shall not be construed as invoking the provisions of 35 U.S.C. §112(6) unless the following claims expressly use the term “means” followed by a particular function without specific structure or expressly use the term “step” followed by a particular function without specific action. All patents and patent applications referred to above are hereby incorporated by reference in their entirety.

1-41. (canceled)

42. A method for delivering at least one marker member to an intracorporeal site within a patient from which tissue has been removed or separated from surrounding tissue, comprising:
a. providing a marker delivery device for delivery of at least one marker member which includes;
i. an elongated shaft which has an inner lumen, a discharge opening in a distal portion of the elongated shaft;
ii. at least one marker member which is formed at least in part of starch or a suitable polysaccharide and which is slidably disposed within the inner lumen of the elongated shaft; and
iii. a plunger element which is slidably disposed within the inner lumen of the marker delivery device proximal to the at least one marker member disposed therein and which is configured to urge the at least one marker member out the discharge opening in the distal portion of the elongated shaft;
b. advancing the marker delivery device within the patient until the distal end of the marker delivery device is disposed at the target tissue site and the discharge opening of the marker delivery device is aligned for marker member deployment; and
c. pressing the plunger element of the marker delivery device to eject the fibrous marker body through the discharge opening in the marker delivery device and into the target site.

43. A method for marking and providing hemostatic activity within a target intracorporeal site within a patient from which tissue has been removed or separated from surrounding tissue, comprising:
a. providing a marker delivery device having at least one marker member which is formed at least in part of starch or suitable polysaccharide and having a discharge port in a distal portion thereof;
b. advancing the marker delivery device within the patient until the discharge port in the distal portion of the marker delivery device is disposed at the target intracorporeal site and the discharge opening of the marker delivery device is aligned for marker member deployment therein; and
c. ejecting the marker member which is formed at least in part of starch or suitable polysaccharide through the discharge opening in the marker delivery device and into the target site.

44. The method of claim 43 wherein the marker delivery device has at least one marker formed at least in part of radiopaque material.

45. The method of claim 44 wherein the marker delivery device has an elongated shaft which has an inner lumen containing the at least one marker member which is formed at least in part of starch or suitable polysaccharide and at least one marker formed at least in part of radiopaque material.

46. The method of claim 43 wherein at least one marker member which is formed at least in part of starch or suitable polysaccharide is formed at least in part of fibrous material.

47. The method of claim 46 wherein the fibrous material is bioabsorbable.

48. The method of claim 46 wherein the fibrous material is formed of strands, has a bulk density of at least 10 mg/cc and an in-vivo life-span of at least three weeks.

49. The method of claim 46 wherein the marker member has a radiopaque element secured thereto.

50. The method of claim 49 wherein the radiopaque element is secured to a central portion of the marker member.

51. The method of claim 50 wherein the radiopaque element is non-magnetic.

52. The method of claim 51 wherein the radiopaque element is formed of a metal selected from the group consisting of titanium, platinum, gold, iridium, tantalum, tungsten, silver, rhodium and non-magnetic stainless steel.

53. The method of claim 46 wherein the marker member is a fibrous mat.

54. The method of claim 53 wherein the fibrous mat is a felt mat.

55. The method of claim 53 wherein the fibrous mat is a woven mat.

56. The method of claim 53 wherein the fibrous mat is a rolled fibrous mat.

57. The method of claim 54 wherein the fibrous mat is a folded fibrous mat.

58. The method of claim 46 wherein the marker member is formed in part of polyglycolic acid.

59. The method of claim 58 wherein the fibrous member is predominantly strands formed of polyglycolic acid.

60. The method of claim 58 wherein the fibrous member is formed of at least 50% by weight of strands of polyglycolic acid.

61. The method of claim 58 wherein the strands of the fibrous member are about 85% to about 100% by weight strands of polyglycolic acid.

62. The method of claim 46 wherein the fibrous member is formed into a shape and at least in part held in the shape by a binding agent.

63. The method of claim 62 wherein the binding agent is a water soluble polymer.

64. The method of claim 46 wherein the fibrous member is about 0.25 to about 1.5 inch long, about 0.25 to about 1.0 wide and about 0.02 to about 0.4 inch thick.

65. The method of claim 56 wherein the rolled fibrous mat is about 0.5 to about 12 mm thick.

66. The method of claim 53,wherein the fibrous mat is about 1 to about 8 mm thick.

67. The method of claim 53 wherein the fibrous mat is about 5 to about 30 mm in length.

68. The method of claim 53 wherein the fibrous mat is about 10 to about 25 mm in length.

69. The method of claim 50 wherein the radiopaque element at least in part surrounds a central portion of the fibrous body.

70. The method of claim 46 wherein the fibrous body has a bulk density of about 30 to about 100 mm/cc.

71. The method of claim 46 wherein the fibrous body is compressed before shaping.

72. The method of claim 46 wherein the fibrous body is compressed after shaping.

73. The method of claim 46 wherein the fibrous body is bound after being compressed and shaped.

74. The method of claim 44 wherein at least one marker member formed at least in part of starch or a suitable polysaccharide is in a pellet form.

75. The method of claim 44 wherein a plurality of marker members are in the form of pellets.

76. The method of claim 75 wherein a plurality of the marker members in the form of pellets contain starch or a suitable polysaccharide.

77. The method of claim 43 wherein at least one of the marker members formed at least in part of starch or a suitable polysaccharide is disposed between two marker members which do not contain starch or a suitable polysaccharide.
